(19) United States
(12) Patent Application Publication
(10) Pub. No.: US 2025/0037866 A1
(43) Pub. Date: Jan. 30, 2025

(54) METHOD FOR PREDICTION OF ALZHEIMER'S DISEASE-RELATED BIOMARKERS

(71) Applicant: KOREA UNIVERSITY RESEARCH AND BUSINESS FOUNDATION, Seoul (KR)

(72) Inventors: Yeong Hun SONG, Seoul (KR); Jun Kyung SEONG, Seoul (KR); Wha jin LEE, Seoul (KR)

(73) Assignee: KOREA UNIVERSITY RESEARCH AND BUSINESS FOUNDATION, Seoul (KR)

(21) Appl. No.: 18/785,803

(22) Filed: Jul. 26, 2024

(30) Foreign Application Priority Data
Jul. 27, 2023 (KR) 10-2023-0098294

(51) Int. Cl. G16H 50/20; G16H 10/60; G16H 30/40; G16H 50/70

(52) U.S. Cl. CPC G16H 50/20; G16H 10/60; G16H 30/40; G16H 50/70

(57) ABSTRACT

The method for predicting Alzheimer's disease-related biomarkers according to the present disclosure involves a method executed by one or more processors of a computing device. The method comprises collecting medical images of a subject, preprocessing the collected medical images to generate training data, training a biomarker prediction model using the generated training data and the subject's demographic and clinical information, where the training includes assigning weights to the training data, and predicting Alzheimer's disease-related biomarkers for a patient using the trained biomarker prediction model.

### BACKGROUND OF THE INVENTION

### 1. Field of the Invention

The present disclosure relates to a method for predicting global and regional Alzheimer's disease-related biomarkers by utilizing brain magnetic resonance imaging (MRI) in combination with demographic and clinical information.

### 2. Description of the Prior Art

Alzheimer's disease is a chronic neurodegenerative disorder characterized by symptoms such as memory decline, language impairment, and cognitive dysfunction. Neuropathologically, Alzheimer's disease is distinguished by the presence of brain cell and neural tissue deposits, plaques within blood vessels, neurofibrillary tangles (NFT), amyloid plaques formed by amyloid peptides, tau proteins, and synaptic damage. The cause of Alzheimer's disease is not fully understood, and there is currently no known cure. Alzheimer's disease is the most common form of dementia and, alongside cardiovascular diseases and cancer, is a leading cause of death. As the average human lifespan increases, the prevalence of Alzheimer's disease is also expected to rise.

Traditionally, the diagnosis of Alzheimer's disease has relied on measuring the accumulation of Alzheimer's disease-related biomarkers, such as amyloid and tau, using positron emission tomography (PET) or by lumbar puncture (LP) to analyze cerebrospinal fluid (CSF). However, PET imaging poses concerns due to the potential side effects from the injection of radioactive tracers into the body, and it also imposes a high financial burden on patients. The lumbar puncture method is invasive and can cause significant discomfort to the patient.

Given this background, there is a growing need for the development of non-invasive and cost-effective methods to measure or predict the accumulation of Alzheimer's disease-related biomarkers in various brain regions.

The information disclosed in this Background of the Invention section is only for enhancement of understanding of the general background of the invention and may not be taken as an acknowledgement or any form of suggestion that this information forms the prior art already known to a person skilled in the art.

### SUMMARY OF THE INVENTION

The present disclosure aims to propose a method for predicting global amyloid positivity/negativity, as well as the degree of amyloid and tau accumulation, using individual patient data such as diffusion-weighted imaging, T1-weighted magnetic resonance imaging, along with demographic and clinical information. This method does not require the direct measurement of Alzheimer's disease-related biomarker accumulation values through positron emission tomography (PET) or from cerebrospinal fluid (CSF) obtained by lumbar puncture (LP).

The method for predicting Alzheimer's disease-related biomarkers according to the present disclosure includes the following steps, executed by one or more processors of a computing device, of: collecting medical images of a subject and preprocessing the collected medical images to generate training data; training a biomarker prediction model using the generated training data and demographic and clinical information of the subject, wherein the training includes assigning weights to the training data; and predicting the Alzheimer's disease-related biomarkers of a patient using the trained biomarker prediction model.

The step of predicting the Alzheimer's disease-related biomarkers may include predicting the amyloid positivity or negativity in the entire brain or in specific brain regions, or predicting the degree of tau accumulation in specific brain regions.

The step of training the biomarker prediction model may include analyzing the correlation between the values derived from diffusion-weighted imaging and the cortical thickness in specific brain regions with the global amyloid and tau values, respectively, and training the weights of each of the diffusion-weighted imaging-derived values and the cortical thickness in specific brain regions based on the analysis results.

The diffusion-weighted imaging-derived values may include at least one of fractional anisotropy (FA) and mean diffusivity (MD) obtained from diffusion-weighted imaging.

The step of training the biomarker prediction model may include extracting diffusion-weighted imaging-derived values related to biomarkers through connectivity-based clustering from diffusion-weighted imaging.

The step of extracting the diffusion-weighted imaging-derived values may include calculating the correlation coefficient between the diffusion-weighted imaging and the biomarkers, selecting regions with a correlation coefficient greater than a specific threshold, and performing connectivity-based clustering by grouping connected voxels using their relationships.

The step of performing connectivity-based clustering may include performing connectivity-based clustering by permuting the order between the diffusion-weighted imaging and the biomarkers, generating a null distribution using the number of voxels in the group with the most abundant voxels as a statistic, performing connectivity-based clustering without permuting the order between the diffusion-weighted imaging and the biomarkers, extracting the values of the voxels belonging to significant groups using the null distribution for each group, and calculating the weighted sum of the extracted voxel values to create a score and training the weights to maximize the correlation coefficient between the created score and the biomarkers.

The step of predicting the Alzheimer's disease-related biomarkers may include calculating a biomarker-specific score using the diffusion-weighted imaging-derived values, the cortical thickness in specific brain regions, and the learned weights, and predicting the positivity or negativity of amyloid in specific regions or globally using the calculated biomarker-specific score along with the demographic and clinical information.

The step of training the biomarker prediction model may include deriving the degree of correlation between the tau accumulation in specific regions at an earlier time point (baseline) and the cortical thickness in all individual regions at a later time point (follow-up) through partial correlation analysis.

The step of deriving the degree of correlation through partial correlation analysis may include deriving the degree of correlation after adjusting for the effects of each variable using demographic and clinical information as covariates.

The step of deriving the degree of correlation through partial correlation analysis may include deriving the degree of correlation according to the progression of the disease, selecting regions that exhibit the most consistent most correlation with respect to the regions where tau accumulation is to be estimated, and conducting multi-output regression analysis based on the selected regions.

The biomarker prediction model may include a first prediction model that predicts the degree of tau accumulation at a previous time point (baseline) based on the cortical thickness in the selected regions at a later time point (follow-up), along with demographic and clinical information.

The biomarker prediction model may further include a second prediction model that predicts the degree of tau accumulation at the later time point (follow-up) based on the cortical thickness at the later time point, the demographic and clinical information, and the degree of tau accumulation at the previous time point (baseline).

The step of predicting Alzheimer's disease-related biomarkers may include predicting the degree of tau accumulation at a past time point using the cortical thickness at the current time point and the demographic and clinical information.

The step of predicting Alzheimer's disease-related biomarkers may further include predicting the degree of tau accumulation at the current time point using the cortical thickness at the current time point, the demographic and clinical information, and the predicted degree of tau accumulation at the past time point.

The computing device of the present disclosure includes a processor with one or more cores and memory. The processor is configured to collect medical images of a subject, preprocess the collected medical images to generate training data, train a biomarker prediction model that includes weights for the training data using the generated training data along with the subject's demographic and clinical information, and predict Alzheimer's disease-related biomarkers of the patient using the trained biomarker prediction model.

The computer-readable storage medium of the present disclosure stores a computer program that includes instructions to cause a computer to perform the following operations of: collecting medical images of a subject and preprocessing the collected medical images to generate training data; training a biomarker prediction model that includes weights for the training data using the generated training data along with the subject's demographic and clinical information; and predicting Alzheimer's disease-related biomarkers of the patient using the trained biomarker prediction model.

Provided according to the present disclosure is an environment in which Alzheimer's disease-related biomarkers can be predicted using MRI images without the need to directly measure the accumulation values of these biomarkers through positron emission tomography (PET) or lumbar puncture (LP) in cerebrospinal fluid (CSF).

Additionally, the present disclosure provides a non-invasive and cost-effective environment for predicting the accumulation of Alzheimer's disease-related biomarkers, as the present disclosure does not require the use of PET imaging or cerebrospinal fluid testing, thereby avoiding additional costs or discomfort for the patient.

Furthermore, the present disclosure provides an environment that allows for the prediction of not only global amyloid positivity/negativity but also the degree of tau accumulation in specific regions, which can assist in estimating the progression status of Alzheimer's disease in individuals.

Moreover, the present disclosure provides an environment that can aid in the early diagnosis and prognosis of Alzheimer's disease by predicting a degree of amyloid and tau accumulation not only in the stages of mild cognitive impairment (MCI) or Alzheimer's disease where the disease is practically progressing, but also in the preclinical and normal aging stages.

### BRIEF DESCRIPTION OF THE DRAWINGS

The above and other aspects, features and advantages of the present disclosure will be more apparent from the following detailed description taken in conjunction with the accompanying drawings, in which:

FIG. 1 is a block diagram of a computing device for predicting Alzheimer's disease-related biomarkers according to an embodiment of the present disclosure;

FIG. 2 is a diagram briefly illustrating the process of collecting and preprocessing medical images of a subject, determining biomarker characteristics and weights using demographic and clinical information of the subject, and predicting biomarkers according to an embodiment of the present disclosure;

FIG. 3 is a flowchart briefly illustrating the process of deriving correlation coefficients between diffusion-weighted imaging and biomarkers, performing connectivity-based clustering to train weights, and predicting amyloid positivity or negativity in specific regions or globally according to an embodiment of the present disclosure;

FIG. 4 is a plot showing the first result of calculating the area under the receiver operating characteristic curve (AUROC) for predicting global amyloid positivity and negativity according to an embodiment of the present disclosure;

FIG. 5 is a plot showing the second result of calculating the area under the receiver operating characteristic curve (AUROC) for predicting global amyloid positivity and negativity according to an embodiment of the present disclosure;

FIG. 6 is a flowchart illustrating the process of deriving the degree of correlation for each variable through partial correlation analysis, predicting the degree of tau accumulation at a past time point for selected regions with high correlation, and predicting the degree of tau accumulation at the current time point using the degree of tau accumulation at the past time point, according to an embodiment of the present disclosure;

FIG. 7 is a plot illustrating an example of predicting the degree of tau accumulation at a past time point using the cortical thickness at the current time point along with demographic and clinical information, and predicting the degree of tau accumulation at the current time point using the degree of tau accumulation at the past time point, according to an embodiment of the present disclosure;

FIG. 8 is a conceptual diagram showing the network function according to an embodiment of the present disclosure;

FIG. 9 shows diagrams showing the correlation between predicted and actual values of tau accumulation in specific regions according to an embodiment of the present disclosure; and

FIG. 10 is a schematic diagram of the computing environment according to an embodiment of the present disclosure.

### DETAILED DESCRIPTION OF THE EXEMPLARY EMBODIMENTS

Reference will now be made in detail to the preferred embodiments of the present disclosure, examples of which are illustrated in the accompanying drawings. The accompanying drawings, which are included to provide a further understanding of the disclosure, and which are incorporated in and constitute a part of this application, illustrate embodiments of the disclosure and together with the description serve to explain the principle of the invention. Wherever possible, like reference numbers will be used throughout the drawings to refer to like parts. And, parts irrelevant to the description of the present disclosure have been omitted.

Throughout the specification, unless explicitly described to the contrary, the word “comprise” and variations such as “comprises” or “comprising”, will be understood to imply the inclusion of stated elements but not the exclusion of any other elements. Additionally, the terms such as “unit,” “device,” “module,” etc., as used in the specification, refer to units that process at least one function or operation and may be implemented as hardware, software, or a combination of hardware and software.

Various embodiments are now described with reference to the drawings. In the specification, various explanations are provided to aid in the understanding of the disclosure. However, it is evident that these embodiments can be practiced without such specific explanations.

“Component”, “module”, “system”, and the like which are terms used in the specification refer to a computer-related entity, hardware, firmware, software, and a combination of the software and the hardware, or execution of the software. For example, the component may be a processing procedure executed on a processor, the processor, an object, an execution thread, a program, and/or a computer, but is not limited thereto. For example, both an application executed in a computing device and the computing device may be the components. One or more components may reside within the processor and/or a thread of execution. One component may be localized in one computer. One component may be distributed between two or more computers. Further, the components may be executed by various computer-readable media having various data structures, which are stored therein. The components may perform communication through local and/or remote processing according to a signal (for example, data transmitted from another system through a network such as the Internet through data and/or a signal from one component that interacts with other components in a local system and a distribution system) having one or more data packets, for example.

The term “or” is intended to mean not exclusive “or” but inclusive “or”. That is, when not separately specified or not clear in terms of a context, a sentence “X uses A or B” is intended to mean one of the natural inclusive substitutions. That is, the sentence “X uses A or B” may be applied to all of the case where X uses A, the case where X uses B, or the case where X uses both A and B. Further, it should be understood that the term “and/or” used in the specification designates and includes all available combinations of one or more items among enumerated related items.

It should be appreciated that the term “comprise” and/or “comprising” means presence of corresponding features and/or components. However, it should be appreciated that the term “comprises” and/or “comprising” means that presence or addition of one or more other features, components, and/or a group thereof is not excluded. Further, when not separately specified or it is not clear in terms of the context that a singular form is indicated, it should be construed that the singular form generally means “one or more” in the present specification and the claims.

The term “at least one of A or B” should be interpreted as encompassing “A only,” “B only,” or “both A and B.”

Those skilled in the art need to additionally recognize that various illustrative logical blocks, configurations, modules, circuits, means, logic, and algorithm steps described in connection with the exemplary embodiments disclosed herein may be implemented as electronic hardware, computer software, or combinations of both sides. To clearly illustrate the interchangeability of hardware and software, various illustrative components, blocks, structures, means, logic, modules, circuits, and steps have been described above generally in terms of their functionalities. Whether the functionalities are implemented as the hardware or software depends on a specific application and design restrictions given to an entire system. Skilled artisans may implement the described functionalities in various ways for each particular application. However, such implementation decisions should not be interpreted as causing a departure from the scope of the present disclosure.

The description of the presented exemplary embodiments is provided so that those skilled in the art of the present disclosure use or implement the present disclosure. Various modifications to the exemplary embodiments will be apparent to those skilled in the art. Generic principles defined herein may be applied to other embodiments without departing from the scope of the present disclosure. Therefore, the present disclosure is not limited to the exemplary embodiments presented herein. The present disclosure should be analyzed within the widest range which is coherent with the principles and new features presented herein.

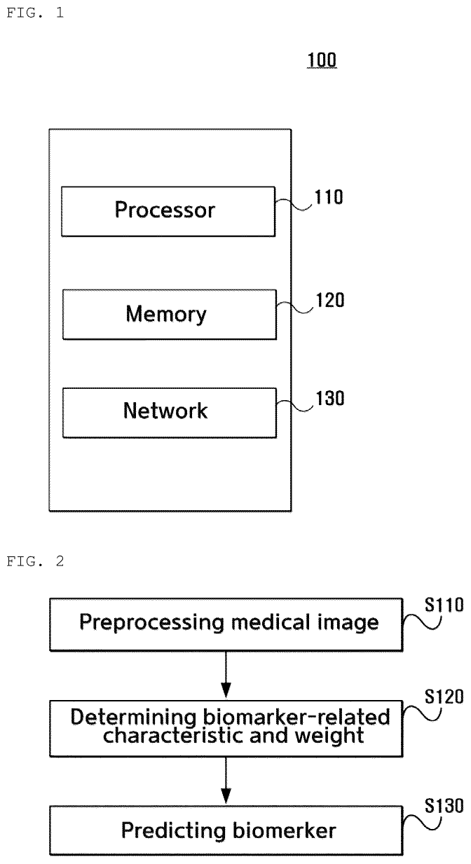

The method for predicting Alzheimer's disease-related biomarkers according to an embodiment of the present disclosure will now be described in detail with reference to FIGS. 1 to 10.

FIG. 1 is a block diagram of a computing device for predicting Alzheimer's disease-related biomarkers according to an embodiment of the present disclosure.

The configuration of the computing device 100 shown in FIG. 1 is a simplified example. In an embodiment of the present disclosure, the computing device 100 may include other configurations necessary to perform the computing environment of the computing device 100, and only some of the disclosed configurations may constitute the computing device 100.

According to an embodiment of the present disclosure, the computing device 100 may include a processor 110, memory 120, and a network 130.

The processor 110 may be composed of one or more cores and may include processors for data analysis and deep learning, such as a central processing unit (CPU), a general-purpose graphics processing unit (GPGPU), or a tensor processing unit (TPU). The processor 110 may read a computer program stored in memory 120 to perform data processing for machine learning according to an embodiment of the present disclosure. According to an embodiment of the present disclosure, the processor 110 may perform calculations for learning the neural network, which include processing of input data for learning in deep learning (DL), extracting features from the input data, calculating errors, and updating neural network weights using backpropagation. At least one of the CPU, GPGPU, and TPU of the processor 110 may process the learning of network functions. For example, the CPU and GPGPU may jointly handle the learning of network functions and the classification of data using the network functions. Additionally, in an embodiment of the present disclosure, the processors of multiple computing devices may be used together to handle the learning of network functions and data classification using the network functions. The computer program executed by the computing device in accordance with an embodiment of the present disclosure may be executable by the CPU, GPGPU, or TPU.

According to an embodiment of the present disclosure, the processor 110 may collect medical images of a subject, preprocess the collected medical images to generate training data, train a biomarker prediction model that includes weights for the training data using the generated training data and the subject's demographic and clinical information, and predict Alzheimer's disease-related biomarkers for the subject using the trained biomarker prediction model.

Here, the medical images may include at least one of T1-weighted magnetic resonance imaging (MRI), diffusion-weighted imaging (DWI), T2-weighted MRI, or susceptibility-weighted imaging (SWI). The training data may include at least one of cortical thickness in specific brain regions derived from T1-weighted MRI, diffusion-weighted imaging-derived values from DWI, vascular condition derived from T2-weighted MRI, or the degree of iron accumulation derived from SWI.

The demographic and clinical information may include demographic information such as age, gender, and years of education, or clinical information such as Mini-Mental State Examination (MMSE) scores.

The processor 110 may predict amyloid positivity or negativity in specific brain regions or globally, or predict the degree of tau accumulation in specific brain regions.

The processor 110 may calculate the correlation coefficients (e.g., Kendall's tau) between the diffusion-weighted imaging-derived values and cortical thickness in specific regions with the global amyloid and tau values, respectively, and train the weights for each of the diffusion-weighted imaging-derived values and the cortical thickness in specific regions to maximize these correlation coefficients.

Here, the diffusion-weighted imaging-derived values may include fractional anisotropy (FA) and mean diffusivity (MD) derived from diffusion-weighted imaging.

The processor 110 can extract characteristics related to biomarkers from diffusion-weighted imaging (DWI) through connectivity-based clustering. The processor 110 may calculate the correlation coefficient between the DWI and the biomarkers and select regions where the correlation coefficient exceeds a specific threshold. The correlation coefficient between the DWI and the biomarkers may include various types of correlation coefficients, such as Kendall's tau, Pearson's correlation coefficient, and Spearman's rank correlation coefficient.

Additionally, the processor 110 can perform connectivity-based clustering by grouping connected voxels based on the relationships between them.

Here, diffusion-weighted imaging (DWI) is a type of magnetic resonance imaging (MRI) that measures the diffusion of water molecules within tissue, based on the principle that water molecules diffuse more easily in certain directions depending on the microstructural environment of the tissue. By measuring the direction of water molecule diffusion through DWI, fractional anisotropy (FA) can be calculated, allowing inferences about the microstructure of the tissue.

Fractional anisotropy (FA) represents the degree of anisotropy (directional dependence) of water diffusion within tissue and can be used as an indicator of tissue microstructure in DWI, a type of MRI that measures water diffusion within tissue.

Connectivity-based clustering is a clustering method based on the principle that objects that are close together are more related to each other than those that are far apart. For example, when an unweighted graph is present, connectivity-based clustering can be performed by grouping connected objects into a single group.

For instance, the processor 110 can perform connectivity-based clustering while permuting the order between the DWI and the biomarkers, creating a null distribution using the number of voxels in the largest group as a statistic.

The processor 110 can then perform connectivity-based clustering without permuting the order between the DWI and the biomarkers, calculate p-values for each group using the null distribution, and extract the values of voxels belonging to significant groups where the p-value is smaller than a set threshold.

The processor 110 can then create a score by weighting and summing the extracted voxel values, and can train the weights to maximize the correlation coefficient (e.g., Kendall's tau) between the created score and the biomarkers.

The processor 110 can calculate a biomarker-specific score using the DWI-derived values, the cortical thickness in specific regions, and the trained weights. Here, the biomarker-specific score includes a quantified score related to Alzheimer's disease-related biomarkers.

Furthermore, the processor 110 can predict amyloid positivity or negativity in specific regions or globally using the calculated biomarker-specific score in combination with the demographic and clinical information.

Additionally, the processor 110 can derive the degree of correlation between the tau accumulation in specific regions at an earlier time point (baseline) and the cortical thickness in all individual regions at a later time point (follow-up) through partial correlation analysis.

For example, the processor 110 can derive the degree of correlation after adjusting for the effects of each variable by using demographic and clinical information as covariates.

The processor 110 can derive the degree of correlation based on the progression of the disease, select the regions with the most consistent correlation for estimating the degree of tau accumulation in each region, and perform multi-output regression analysis based on the selected regions.

The processor 110 can train the biomarker prediction model, which includes the weights of the training data, using the training data generated by preprocessing the medical images and the demographic and clinical information of the subject.

Here, the biomarker prediction model may include a first prediction model that predicts the degree of tau accumulation at a previous time point (baseline) based on the cortical thickness of the selected regions at a later time point (follow-up) and the demographic and clinical information, and a second prediction model that predicts the degree of tau accumulation at the later time point (follow-up) based on the cortical thickness at the later time point, the demographic and clinical information, and the degree of tau accumulation at the previous time point (baseline).

The processor 110 can predict the degree of tau accumulation at a past time point using the cortical thickness at the current time point and the demographic and clinical information.

Additionally, the processor 110 can predict the degree of tau accumulation at the current time point using the cortical thickness at the current time point, the demographic and clinical information, and the predicted degree of tau accumulation at the past time point.

According to an embodiment of the present disclosure, the memory 120 may store any form of information generated or determined by the processor 110, as well as any form of information received from the network 130.

The memory 120, according to an embodiment of the present disclosure, may include at least one type of storage medium such as flash memory, hard disk type, multimedia card micro type, card type memory (e.g., SD or XD memory), Random Access Memory (RAM), Static Random Access Memory (SRAM), Read-Only Memory (ROM), Electrically Erasable Programmable Read-Only Memory (EEPROM), Programmable Read-Only Memory (PROM), magnetic memory, magnetic disk, or optical disk. The computing device 100 may also operate in conjunction with web storage that performs the storage function of the memory 120 over the Internet. The description of the memory types above is illustrative and not limiting.

The network 130, according to an embodiment of the present disclosure, may utilize any form of known wired or wireless communication system.

The network 130 can receive MR images of the subject and other data from related devices or systems.

Additionally, the network 130 can transmit and receive information processed by the processor 110, user interfaces, and other data through communication with other terminals. For example, the network 130 can provide a user interface generated by the processor 110 to a client (e.g., user terminal). The network 130 can also receive external input from an authorized user via the client and transmit it to the processor 110. In this case, the processor 110 can handle operations such as the output, modification, change, or addition of the information provided through the user interface based on the external input received from the network 130.

Meanwhile, the computing device 100 according to an embodiment of the present disclosure may include a server as a computing system that transmits and receives information through communication with a client. In this case, the client may be any type of terminal that can access the server.

In additional embodiments, the computing device 100 may also include any type of terminal that receives data resources generated by any server and performs additional information processing.

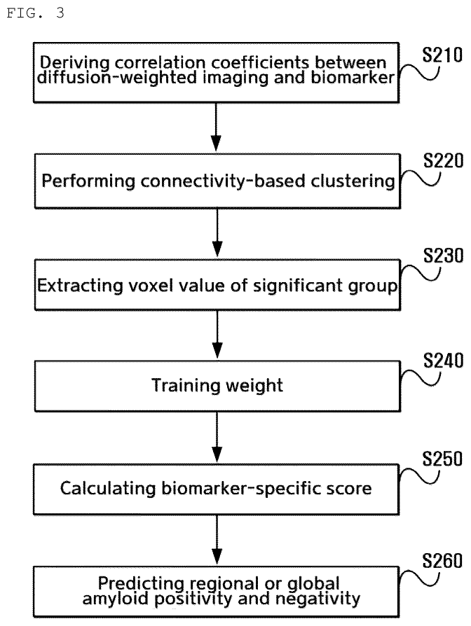

FIG. 2 is a diagram that briefly illustrates the process of collecting and preprocessing medical images of a subject, determining biomarker characteristics and weights using the subject's demographic and clinical information, and then predicting biomarkers according to an embodiment of the present disclosure.

Referring to FIG. 2, the computing device 100 can collect medical images of the subject and preprocess the collected medical images to generate training data (S110).

The computing device 100 can receive diffusion-weighted imaging (DWI) images, T1-weighted magnetic resonance imaging (MRI) images, and amyloid or tau PET images as inputs.

In the case of T1-weighted MRI images, linear registration methods can be used to align them to a standard brain space, followed by the use of segmentation models to divide the brain into regions such as white matter and cortical areas. The cortical thickness or diameter (hereinafter referred to as cortical thickness) can then be measured using quantification tools.

The computing device 100 can calculate the average cortical thickness for each cortical region and determine a nonlinear registration matrix for aligning the T1-weighted MRI images to the standard brain space.

For DWI images, after performing tensor fitting, values such as fractional anisotropy (FA) can be calculated. The computed values can then be aligned to the space of the T1-weighted MRI images using linear registration methods, and subsequently aligned to the standard brain space using the previously determined nonlinear registration matrix of the T1-weighted MRI images.

PET images can be aligned to the T1-weighted MRI of the same subject using linear registration methods, and standardized uptake value (SUV) can be extracted for each brain region. The standard uptake value ratio (SUVR) can be calculated as a ratio to the SUV of a reference region.

The computing device 100 may normalize the biomarker data for each region so that they are reflected with equal importance. In the present disclosure, the SUVR values of tau can be normalized by converting them to Z-scores based on the amyloid-negative control group values. The Z-score increases in the positive direction as the degree of biomarker accumulation increases compared to the control group.

The computing device 100 can then determine biomarker-related characteristics and weights using the training data generated from the preprocessed medical images (S120).

For example, the computing device 100 can train a biomarker prediction model that includes the weights of the training data, using the generated training data along with the demographic and clinical information of the subject.

Finally, the computing device 100 can predict Alzheimer's disease-related biomarkers of the subject using the trained biomarker prediction model (S130).

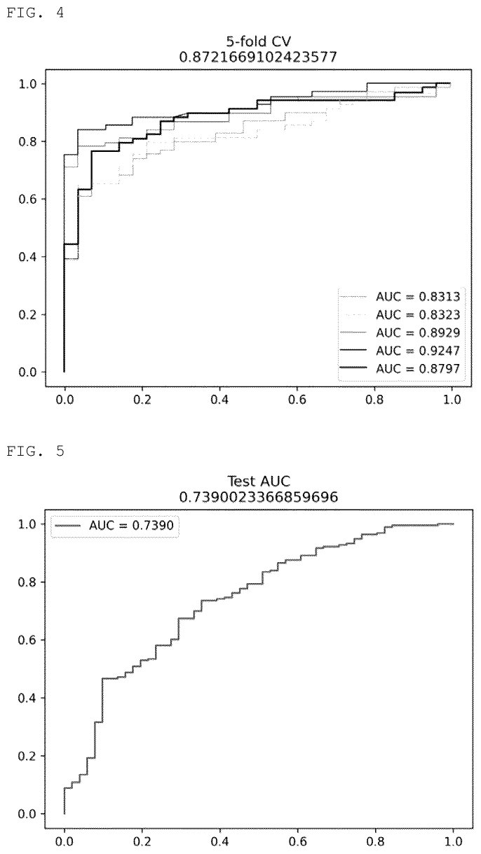

FIG. 3 is a flowchart that briefly illustrates the process of deriving correlation coefficients between diffusion-weighted imaging (DWI) and biomarkers, performing connectivity-based clustering to train weights, and predicting amyloid positivity or negativity in specific regions or globally, according to an embodiment of the present disclosure.

Referring to FIG. 3, the computing device 100 can derive the correlation coefficients between the diffusion-weighted imaging (DWI) and the biomarkers (S210). For example, these correlation coefficients can include various types of correlation coefficients such as Kendall's tau, Pearson's correlation coefficient, and Spearman's rank correlation coefficient.

The computing device 100 can calculate the correlation coefficients (e.g., Kendall's tau) between the diffusion-weighted imaging-derived values and cortical thickness in specific regions with the global amyloid and tau values. Here, the diffusion-weighted imaging-derived values may include fractional anisotropy (FA) and mean diffusivity (MD) derived from DWI.

The computing device 100 can extract characteristics related to the biomarkers from DWI through connectivity-based clustering (S220).

The computing device 100 can calculate the correlation coefficients (e.g., Kendall's tau) between the DWI and the biomarkers, and select regions where the Kendall's tau correlation coefficient exceeds a specific threshold. The computing device 100 can then perform connectivity-based clustering by grouping connected voxels based on their relationships.

The computing device 100 can perform connectivity-based clustering while permuting the order between the DWI and the biomarkers, and create a null distribution using the number of voxels in the largest group as a statistic.

Next, the computing device 100 can perform connectivity-based clustering without permuting the order between the DWI and the biomarkers, calculate p-values for each group using the null distribution, and extract the values of voxels belonging to significant groups where the p-value is smaller than a set threshold (e.g., 0.05) (S230).

Afterward, the computing device 100 can create a score by weighting and summing the extracted voxel values, and train the weights to maximize the correlation coefficient (e.g., Kendall's tau) between the created score and the biomarkers (S240). Here, the computing device 100 can use the entire set of cortical thickness values for each region to train the weights.

Additionally, the computing device 100 can calculate a biomarker-specific score using the diffusion-weighted imaging-derived values, the cortical thickness in specific regions, and the trained weights (S250).

The computing device 100 can then predict amyloid positivity or negativity in specific regions or globally using the calculated biomarker-specific score and the demographic and clinical information (S260). Here, the demographic and clinical information may include demographic information such as age, gender, and years of education, or clinical information such as Mini-Mental State Examination (MMSE) scores.

FIG. 4 is a plot showing the first result of calculating the area under the receiver operating characteristic curve (AUROC) for predicting global amyloid positivity and negativity according to an embodiment of the present disclosure.

Referring to FIG. 4, the computing device 100, according to an embodiment of the present disclosure, conducted an experiment to determine global amyloid positivity or negativity using 5-fold cross-validation. The experiment utilized pairs of diffusion-weighted imaging (DWI), T1-weighted magnetic resonance imaging (MRI), and demographic and clinical information of subjects. The demographic information included age, gender, and years of education, while the clinical information included Mini-Mental State Examination (MMSE) scores.

As a result of calculating the area under the receiver operating characteristic curve (AUROC), which is used as a performance evaluation method for binary classifiers, the computing device 100, according to an embodiment of the present disclosure, obtained an average value of 0.872. This result indicates that the computing device 100, according to an embodiment of the present disclosure, can accurately distinguish between amyloid positivity and negativity across the brain globally.

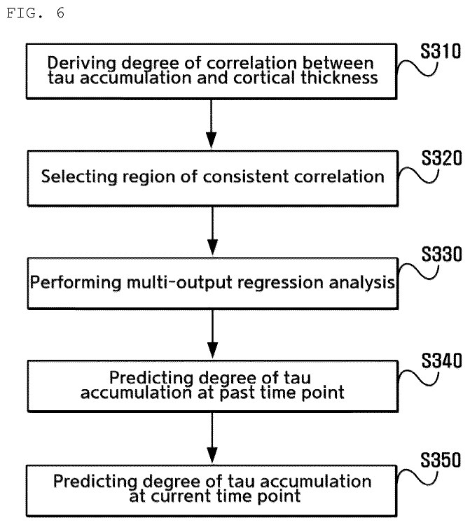

FIG. 5 is a plot showing the second result of calculating the area under the receiver operating characteristic curve (AUROC) for predicting global amyloid positivity and negativity according to an embodiment of the present disclosure.

Referring to FIG. 5, the computing device 100, according to an embodiment of the present disclosure, conducted an experiment to determine global amyloid positivity or negativity by dividing pairs of diffusion-weighted imaging (DWI), T1-weighted magnetic resonance imaging (MRI), and demographic and clinical information of subjects into training and validation datasets. The demographic information included age, gender, and years of education, while the clinical information included Mini-Mental State Examination (MMSE) scores.

In this experiment, the computing device 100, according to an embodiment of the present disclosure, calculated the area under the receiver operating characteristic curve (AUROC), which is used as a performance evaluation method for binary classifiers. The result yielded a value of 0.739, indicating that the computing device 100, according to an embodiment of the present disclosure, can accurately distinguish between amyloid positivity and negativity across the brain globally.

FIG. 6 is a flowchart illustrating the process of deriving the degree of correlation for each variable through partial correlation analysis, predicting the degree of tau accumulation at a past time point for selected regions with high correlation, and predicting the degree of tau accumulation at the current time point using the degree of tau accumulation at the past time point, according to an embodiment of the present disclosure.

Referring to FIG. 6, the computing device 100 can derive the degree of correlation between the tau accumulation in specific regions at a previous time point (baseline) and the cortical thickness in all individual regions at a later time point (follow-up) (S310). For example, the computing device 100 can perform partial correlation analysis to derive the degree of correlation between the tau accumulation in specific regions at the baseline and the cortical thickness in all individual regions at the follow-up, based on the temporal difference between T (tau) and N (neurodegeneration) in the A/T/N framework.

Here, the A/T/N framework classifies the biomarkers of Alzheimer's disease (AD) into beta-amyloid (A), abnormal tau (T), and neurodegeneration (N), and staging of Alzheimer's disease (AD) can be determined solely based on these biomarkers.

Furthermore, the computing device 100 can use demographic and clinical information as covariates during partial correlation analysis to derive the degree of correlation after adjusting for the effects of each variable.

The computing device 100 can derive the degree of correlation according to the progression of the disease and select the regions that consistently show the highest correlation based on the regions where the degree of tau accumulation is to be estimated (S320).

The computing device 100 can then perform multi-output regression analysis using a regressor chain with the selected regions (S330).

In this case, the computing device 100 can train two models simultaneously: a model (Model 1) that predicts the degree of tau accumulation at the baseline using the cortical thickness at the follow-up and the demographic and clinical information of the selected regions, and a model (Model 2) that predicts the degree of tau accumulation at the follow-up based on the inputs of Model 1 and the degree of tau accumulation at the baseline.

Finally, the computing device 100 can predict Alzheimer's disease-related biomarkers for the subject using the trained biomarker prediction model.

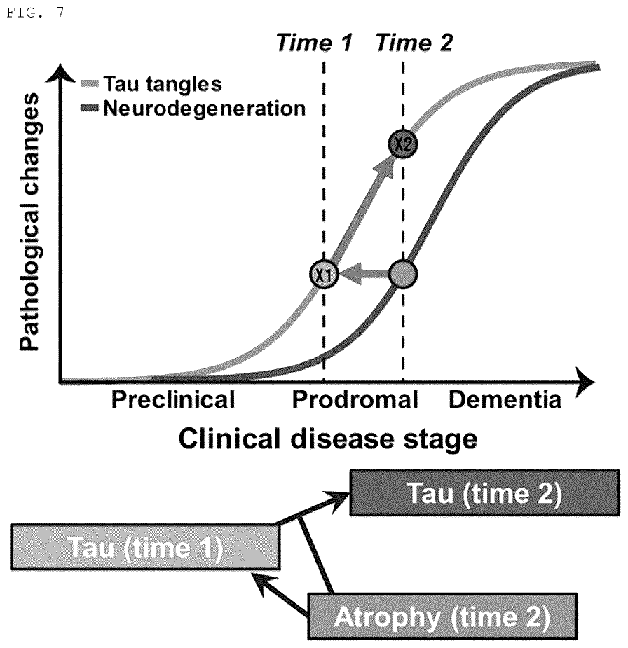

FIG. 7 is a diagram illustrating an example of predicting the degree of tau accumulation at a past time point using the cortical thickness at the current time point and the demographic and clinical information, and then using the degree of tau accumulation at the past time point to predict the degree of tau accumulation at the current time point, according to an embodiment of the present disclosure.

Referring to FIG. 7, the computing device 100 can predict the degree of tau accumulation at a past time point (e.g., X1) by taking the current cortical thickness and the demographic and clinical information of highly correlated selected regions for each tau prediction area as input (S340).

Additionally, the computing device 100 can then predict the degree of tau accumulation at the current time point (e.g., X2) by taking the original input values along with the predicted degree of tau accumulation at the past time point as input (S350).

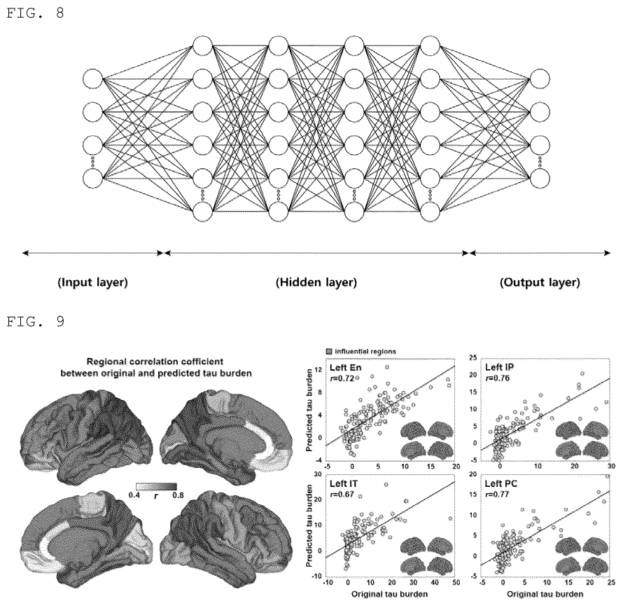

FIG. 8 is a conceptual diagram illustrating the network function according to an embodiment of the present disclosure.

In an embodiment of the present disclosure, the biomarker prediction model (machine learning model or neural network model) may include a neural network designed to predict Alzheimer's disease-related biomarkers from MRI images of a subject.

Throughout this specification, the terms “neural network,” and “network function,” are used interchangeably. A neural network is generally composed of a set of interconnected computational units, which can be referred to as nodes. These nodes may also be called neurons. A neural network is configured with at least one or more nodes, and the nodes (or neurons) that make up the neural network can be interconnected by one or more links.

Within a neural network, one or more nodes connected by links can form relative relationships between input nodes and output nodes. The concepts of input and output nodes are relative; any node that is in an output node relationship with one node may be in an input node relationship with another node, and vice versa. As described above, the input node to output node relationship can be formed around a link. One or more output nodes can be connected to an input node via links, and vice versa.

In the relationship between input and output nodes connected via a link, the data of the output node can be determined based on the data input to the input node. The link that interconnects the input and output nodes may have a weight. This weight can be variable and may be adjusted by the user or the model to perform the desired function of the neural network. For example, when one or more input nodes are interconnected with an output node via respective links, the output node can determine its value based on the values input to the connected input nodes connected to the output node and the weights assigned to the links corresponding to those input nodes.

As described above, a neural network is formed by interconnecting one or more nodes via one or more links, establishing relationships between input and output nodes within the neural network. The characteristics of the neural network can be determined by the number of nodes and links within the neural network, the relationships between nodes and links, and the values of the weights assigned to each link. For example, if two neural networks have the same number of nodes and links but different weight values for the links, the two networks can be recognized as different.

A neural network can be composed of a set of one or more nodes. A subset of the nodes in a neural network may form a layer. Some nodes within the neural network may form a layer based on their distance from the initial input node. For example, a set of nodes that are at a distance of n from the initial input node can form the n-th layer. The distance from the initial input node can be defined by the minimum number of links that must be traversed to reach that node from the initial input node. However, this definition of layers is arbitrary for explanatory purposes, and the dimensionality of the layer within the neural network may be defined differently from the above description. For example, the layers of nodes may be defined by their distance from the final output node.

The initial input node may refer to one or more nodes to which data is directly input without passing a link in a relationship with other nodes among the nodes within the neural network. Otherwise, the initial input node may mean nodes having no other input node connected through the links in a relationship between the nodes based on a link within the neural network. Similarly, the final output node may mean one or more nodes having no output node in the relationship with other nodes among the nodes within the neural network. Further, a hidden node may refer to a node, not the initial input node and the final output node, forming the neural network.

In an embodiment of the present disclosure, the neural network may have an equal number of nodes in the input layer and the output layer, with the number of nodes decreasing as the network progresses from the input layer to the hidden layers and then increasing again. In another embodiment of the present disclosure, the neural network may have fewer nodes in the input layer compared to the output layer, with the number of nodes decreasing as the network progresses from the input layer to the hidden layers. In addition, the neural network according to another embodiment of the present disclosure may have more nodes in the input layer than in the output layer, with the number of nodes increasing as the network progresses from the input layer to the hidden layers. Another embodiment of the present disclosure may involve a neural network that combines these configurations.

A deep neural network (DNN) refers to a neural network that includes multiple hidden layers in addition to the input and output layers. Deep neural networks can be used to identify latent structures in data. For example, they can detect latent structures in images, text, videos, speech, and music (e.g., identifying objects in images, determining the content and sentiment of text, understanding the content and sentiment of speech, etc.). A deep neural network (DNN) may refer to a neural network that includes a plurality of hidden layers in addition to the input and output layers. When the deep neural network is used, the latent structures of data may be determined. That is, latent structures of photos, text, video, voice, and music (e.g., what objects are in the photo, what the content and feelings of the text are, what the content and feelings of the voice are) may be determined. The deep neural network may include convolutional neural networks (CNNs), recurrent neural networks (RNNs), autoencoders, generative adversarial networks (GANs), restricted Boltzmann machines (RBMs), deep belief networks (DBNs), Q-networks, U-networks, Siamese networks, etc. The description of the deep neural network described above is just an example and the present disclosure is not limited thereto.

In an exemplary embodiment of the present disclosure, the network function may include the auto encoder. The auto encoder may be a kind of artificial neural network for outputting output data similar to input data. The auto encoder may include at least one hidden layer and odd hidden layers may be disposed between the input and output layers. The number of nodes in each layer may be reduced from the number of nodes in the input layer to an intermediate layer called a bottleneck layer (encoding), and then expanded symmetrical to reduction to the output layer (symmetrical to the input layer) in the bottleneck layer. The auto encoder may perform non-linear dimensional reduction. The number of input and output layers may correspond to a dimension after preprocessing the input data. The auto encoder structure may have a structure in which the number of nodes in the hidden layer included in the encoder decreases as a distance from the input layer increases. When the number of nodes in the bottleneck layer (a layer having a smallest number of nodes positioned between an encoder and a decoder) is too small, a sufficient amount of information may not be delivered, and as a result, the number of nodes in the bottleneck layer may be maintained to be a specific number or more (e.g., half of the input layers or more).

The neural network may be learned in at least one scheme of supervised learning, unsupervised learning, semi supervised learning, or reinforcement learning. The learning of the neural network may be a process in which the neural network applies knowledge for performing a specific operation to the neural network.

The neural network may be learned in a direction to minimize errors of an output. The learning of the neural network is a process of repeatedly inputting learning data into the neural network and calculating the output of the neural network for the learning data and the error of a target and back-propagating the errors of the neural network from the output layer of the neural network toward the input layer in a direction to reduce the errors to update the weight of each node of the neural network. In the case of the supervised learning, the learning data labeled with a correct answer is used for each learning data (i.e., the labeled learning data) and in the case of the unsupervised learning, the correct answer may not be labeled in each learning data. That is, for example, the learning data in the case of the supervised learning related to the data classification may be data in which category is labeled in each learning data. The labeled learning data is input to the neural network, and the error may be calculated by comparing the output (category) of the neural network with the label of the learning data. As another example, in the case of the unsupervised learning related to the data classification, the learning data as the input is compared with the output of the neural network to calculate the error. The calculated error is back-propagated in a reverse direction (i.e., a direction from the output layer toward the input layer) in the neural network and connection weights of respective nodes of each layer of the neural network may be updated according to the back propagation. A variation amount of the updated connection weight of each node may be determined according to a learning rate. Calculation of the neural network for the input data and the back-propagation of the error may constitute a learning cycle (epoch). The learning rate may be applied differently according to the number of repetition times of the learning cycle of the neural network. For example, in an initial stage of the learning of the neural network, the neural network ensures a certain level of performance quickly by using a high learning rate, thereby increasing efficiency and uses a low learning rate in a latter stage of the learning, thereby increasing accuracy.

In learning of the neural network, the learning data may be generally a subset of actual data (i.e., data to be processed using the learned neural network), and as a result, there may be a learning cycle in which errors for the learning data decrease, but the errors for the actual data increase. Overfitting is a phenomenon in which the errors for the actual data increase due to excessive learning of the learning data. For example, a phenomenon in which the neural network that learns a cat by showing a yellow cat sees a cat other than the yellow cat and does not recognize the corresponding cat as the cat may be a kind of overfitting. The overfitting may act as a cause which increases the error of the machine learning algorithm. Various optimization methods may be used in order to prevent the overfitting. In order to prevent the overfitting, a method such as increasing the learning data, regularization, dropout of omitting a part of the node of the network in the process of learning, utilization of a batch normalization layer, etc., may be applied.

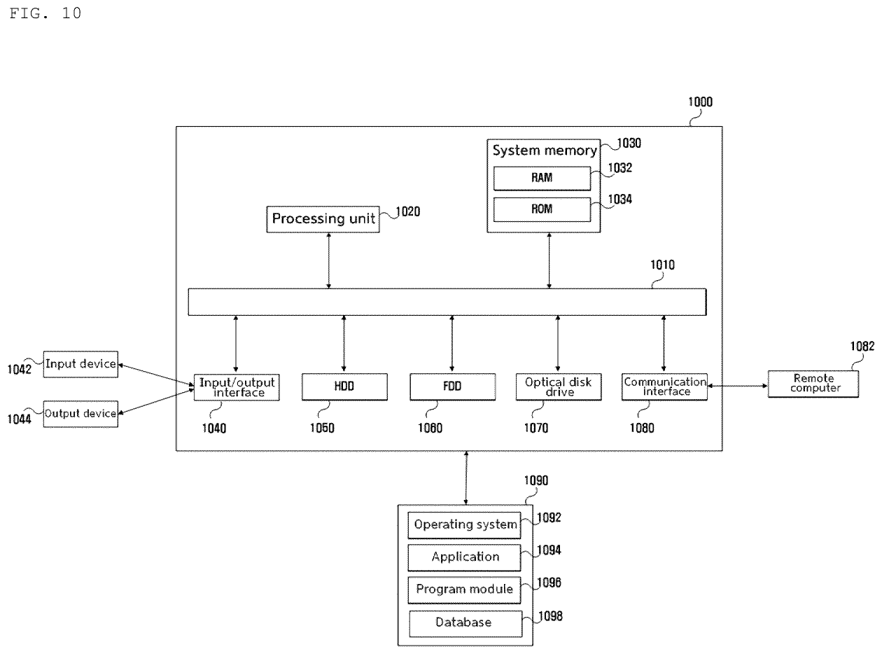

FIG. 9 shows diagrams illustrating the correlation coefficients between the predicted and actual values of tau accumulation in specific regions according to an embodiment of the present disclosure.

Referring to FIG. 9, the computing device 100 conducted an experiment to predict the degree of tau accumulation using 5-fold cross-validation with pairs of T1-weighted magnetic resonance imaging (MRI) and demographic and clinical information obtained from Gangnam Severance Hospital and the ADNI dataset. The demographic information included age, gender, and years of education, while the clinical information included Mini-Mental State Examination (MMSE) scores.

In an embodiment of the present disclosure, the computing device 100 performed a correlation analysis between the actual tau accumulation levels in specific regions and the predicted values to evaluate the performance of the predictions. The analysis yielded a median correlation coefficient of 0.66, with particularly high correlation coefficients observed in certain regions known to be closely related to Alzheimer's disease.

FIG. 10 is a schematic diagram of the computing environment according to an embodiment of the present disclosure.

The present disclosure has been described as being generally implementable by the computing device, but those skilled in the art will appreciate well that the present disclosure is combined with computer executable commands and/or other program modules executable in one or more computers and/or be implemented by a combination of hardware and software.

In general, a program module includes a routine, a program, a component, a data structure, and the like performing a specific task or implementing a specific abstract data form. Further, those skilled in the art will well appreciate that the method of the present disclosure may be carried out by a personal computer, a hand-held computing device, a microprocessor-based or programmable home appliance (each of which may be connected with one or more relevant devices and be operated), and other computer system configurations, as well as a single-processor or multiprocessor computer system, a mini computer, and a main frame computer.

The illustrated embodiments of the present disclosure may also be practiced in distributed computing environments where certain tasks are performed by remote processing devices that are linked through a communications network. In a distributed computing environment, program modules can be located in both local and remote memory storage devices.

The computer generally includes various computer readable media. The computer accessible medium may be any type of computer readable medium, and the computer readable medium includes volatile and non-volatile media, transitory and non-transitory media, and portable and non-portable media. As a non-limited example, the computer readable medium may include a computer readable storage medium and a computer readable transport medium. The computer readable storage medium includes volatile and non-volatile media, transitory and non-transitory media, and portable and non-portable media constructed by a predetermined method or technology, which stores information, such as a computer readable command, a data structure, a program module, or other data. The computer readable storage medium includes a RAM, a Read Only Memory (ROM), an Electrically Erasable and Programmable ROM (EEPROM), a flash memory, or other memory technologies, a Compact Disc (CD)-ROM, a Digital Video Disk (DVD), or other optical disk storage devices, a magnetic cassette, a magnetic tape, a magnetic disk storage device, or other magnetic storage device, or other predetermined media, which are accessible by a computer and are used for storing desired information, but is not limited thereto.

The computer readable transport medium generally implements a computer readable command, a data structure, a program module, or other data in a modulated data signal, such as a carrier wave or other transport mechanisms, and includes all of the information transport media. The modulated data signal means a signal, of which one or more of the characteristics are set or changed so as to encode information within the signal. As a non-limited example, the computer readable transport medium includes a wired medium, such as a wired network or a direct-wired connection, and a wireless medium, such as sound, Radio Frequency (RF), infrared rays, and other wireless media. A combination of the predetermined media among the foregoing media is also included in a range of the computer readable transport medium.

An exemplary environment for implementing various aspects of the present disclosure, including a computer 1000, is shown, where the computer 1000 includes a processing unit 1020, a system memory 1030, and a system bus 1010. The system bus 1010 connects system components, including the system memory 1030 (not limited thereto), to the processing unit 1020. The processing unit 1020 may be any of various commercially available processors. Dual processors and other multi-processor architectures can also be used as the processing unit 1020.

The system bus 1010 may be any of several types of bus structures, which may be additionally connectable to a local bus using a predetermined one among a memory bus, a peripheral device bus, and various common bus architectures. The system memory 1030 includes read-only memory (ROM) 1034 and random access memory (RAM) 1032. A basic input/output system (BIOS), which contains the basic routines that help to transfer information between elements within the computer 1000 during start-up, is stored in non-volatile memory 1034, such as ROM, EPROM, or EEPROM. The RAM 1032 may also include high-speed RAM, such as static RAM, for caching data.

The computer 1000 may also include an embedded hard disk drive (HDD) 1050 (e.g., EIDE, SATA)—this embedded hard disk drive 1050 may also be configured for exterior mounted usage within a proper chassis (not shown)—a magnetic floppy disk drive (FDD) 1060 (e.g., for reading from or writing to a removable diskettes), and an optical disk drive 1070 (e.g., for reading from a CD-ROM disk or reading from or writing to other high-capacity optical media such as a DVD). The hard disk drive 1050, the magnetic disk drive 1060, and the optical disk drive 1070 can each be connected to the system bus 1010 by a hard disk drive interface, a magnetic disk drive interface, and an optical drive interface, respectively. Interfaces for external drive implementations may include at least one or both of Universal Serial Bus (USB) and IEEE 1394 interface technologies.

These drives and their associated computer-readable media provide non-volatile storage of data, data structures, computer-executable instructions, and so forth. In the context of the computer 1000, the drives and media correspond to storing any data in an appropriate digital format. While the description of computer-readable media above refers to HDDs, removable magnetic disks, and removable optical media such as CDs or DVDs, it will be understood by those skilled in the art that other types of media that are readable by a computer, such as zip drives, magnetic cassettes, flash memory cards, cartridges, and the like, may also be used in the exemplary operating environment and may contain computer-executable instructions for performing the methods of the present disclosure.

Numerous program modules, including an operating system 1092, one or more application programs 1094, other program modules 1096, and databases 1098, may be stored on the drives and in RAM 1032. Parts or the entirety of the operating system, applications, modules, and/or data may also be cached in RAM 1032. It will be appreciated that the present disclosure can be implemented in a variety of commercially available operating systems or combinations of operating systems.

A user can input commands and information into the computer 1000 through one or more wired or wireless input devices 1042, such as a keyboard and a pointing device like a mouse. Other input devices (not shown) may include a microphone, IR remote control, joystick, gamepad, stylus pen, touchscreen, and others. These and other input devices are often connected to the processing unit 1020 through an input/output interface 1040 that is connected to the system bus 1010, but they can also be connected via other interfaces such as a parallel port, IEEE 1394 serial port, game port, USB port, IR interface, and others.

A monitor or other type of display device is also connected to the system bus 1010 via an interface, such as a video adapter. In addition to the monitor, computers typically include other peripheral output devices (not shown), such as speakers and printers.

The computer 1000 can operate in a networked environment using logical connections to one or more remote computers 1082 through wired and/or wireless communication. The remote computers 1082 may be workstations, computing devices, personal computers, portable computers, microprocessor-based entertainment devices, peer devices, or other common network nodes, and they typically include many or all of the components described for the computer 1000. The logical connections may include wired and/or wireless connections to a local area network (LAN) and/or a larger network, such as a wide area network (WAN). These LAN and WAN networking environments are common in offices and corporate settings and facilitate enterprise-wide computer networks, such as intranets, all of which may connect to global computer networks like the internet.

When used in a LAN networking environment, the computer 1000 may be connected to the local network (not shown) via a wired and/or wireless communication network interface or adapter (not shown). The adapter may facilitate wired or wireless communication to the LAN (not shown), which may include a wireless access point installed therein to communicate with a wireless adapter (not shown). In a WAN networking environment, the computer 1000 may include a modem (not shown) or be connected to communication computing devices on the WAN (not shown) or have other means for establishing communications over the WAN, such as through the internet. The modem, which may be an embedded or outer-mounted and wired or wireless device, is connected to the system bus 1010 via a serial port interface (not shown). In a networked environment, the program modules or portions thereof described in relation to the computer 1000 may be stored in a remote memory or storage device (not shown). It should be appreciated that the network connections shown are exemplary and that other means of establishing communication links between the computers may be used.

The computer 1000 performs an operation of communicating with a predetermined wireless device or entity, for example, a printer, a scanner, a desktop and/or portable computer, a portable data assistant (PDA), a communication satellite, predetermined equipment or place related to a wirelessly detectable tag, and a telephone, which is disposed by wireless communication and is operated. The operation includes Wi-Fi and Bluetooth wireless technology at least. Accordingly, the communication may have a pre-defined structure, such as a network in the related art, or may be simply ad hoc communication between at least two devices.

Wireless Fidelity (Wi-Fi) enables connections to the internet and other networks without the need for wired connections. Wi-Fi is a wireless technology that allows devices, such as computers, to transmit and receive data anywhere within the range of a base station, both indoors and outdoors, similar to a cellular phone. Wi-Fi networks use wireless technology called IEEE 802.11 (a, b, g, etc.) to provide secure, reliable, and high-speed wireless connections. Wi-Fi can be used to connect computers to each other, to the internet, and to wired networks (using IEEE 802.3 or Ethernet). Wi-Fi networks operate in unlicensed 2.4 and 5 GHz radio bands at data rates of, for example, 11 Mops (802.11a) or 54 Mbps (802.11b), and can operate on products that include dual bands.

Those skilled in the art will appreciate that information and signals can be represented using a variety of different technologies and techniques. For example, data, instructions, commands, information, signals, bits, symbols, and chips that may be referenced in the above description can be represented by voltages, currents, electromagnetic waves, magnetic fields or particles, optical fields or particles, or any combination of these.

Those skilled in the art will also understand that various exemplary logical blocks, modules, processors, means, circuits, and model steps described in relation to the embodiments disclosed herein can be implemented in electronic hardware, various forms of program or design code (conveniently referred to herein as “software”), or combinations of both. To clearly illustrate this interchangeability of f hardware and software, various exemplary components, blocks, modules, circuits, and steps have been described above generally in terms of their functionality. Whether such functionality is implemented as hardware or software depends on the specific application and design constraints imposed on the overall system. Those skilled in the art may implement the described functionality in various ways for each specific application, but such implementation decisions should not be interpreted as limiting the scope of the present disclosure.

The various embodiments presented herein can be implemented as methods, devices, or articles of manufacture using standard programming and/or engineering techniques. The term “article of manufacture” includes any computer-readable storage device that can be accessed by a computer program, carrier, or medium. For example, computer-readable storage media include magnetic storage devices (e.g., hard disks, floppy disks, magnetic strips, etc.), optical discs (e.g., CDS, DVDS, etc.), smart cards, and flash memory devices (e.g., EEPROM, cards, sticks, key drives, etc.), but are not limited to these. Additionally, the various storage media presented herein may include one or more devices and/or other machine-readable media for storing information.

It should be understood that the specific order or hierarchy of steps in the processes presented is an example of an illustrative approach. Based on design preferences, the specific order or hierarchy of steps in the processes may be rearranged within the scope of the present disclosure. The appended method claims provide the elements of the various steps in a sample order but are not meant to be limited to the specific order or hierarchy presented.

The description of the embodiments presented is provided so that any person skilled in the art may utilize or implement the present disclosure. Various modifications to these embodiments will be apparent to those skilled in the art, and the general principles defined herein may be applied to other embodiments without departing from the scope of the present disclosure. Thus, the present disclosure is not intended to be limited to the embodiments presented herein, but should be accorded the broadest scope consistent with the principles and novel features disclosed herein.

The embodiments of the present invention described above can be implemented not only through devices and methods but also through a program that realizes the functions corresponding to the configurations of the embodiments of the present invention, or through a recording medium on which the program is recorded. Such a recording medium can be executed on both servers and user terminals.

Although the embodiments of the present invention have been described in detail above, the scope of the present invention is not limited to these, and various modifications and improvements using the basic concepts defined in the following claims by those skilled in the art also fall within the scope of the present invention.

What is claimed is:

1. A method for predicting Alzheimer's disease-related biomarkers, executable by one or more processors of a computing device, the method comprising the steps of:
collecting medical images of a subject and preprocessing the collected medical images to generate training data;
training a biomarker prediction model using the generated training data and demographic and clinical information of the subject, wherein the training includes assigning weights to the training data; and
predicting Alzheimer's disease-related biomarkers for a patient using the trained biomarker prediction model.

2. The method of claim 1, wherein the step of predicting Alzheimer's disease-related biomarkers comprises:
predicting regional or global amyloid positivity or negativity in the brain, or predicting a degree of tau accumulation in specific brain regions.

3. The method of claim 1, wherein the step of training a biomarker prediction model comprises:
analyzing correlation between diffusion-weighted imaging-derived values and regional cortical thickness with global amyloid and tau values, respectively, and
training the model by assigning weights to the diffusion-weighted imaging-derived values and the regional cortical thickness based on the analysis.

4. The method of claim 3, wherein the diffusion-weighted imaging-derived values comprise at least one of fractional anisotropy (FA) and mean diffusivity (MD) derived from diffusion-weighted imaging.

5. The method of claim 1, wherein the step of training the biomarker prediction model comprises:
extracting diffusion-weighted imaging-derived values related to biomarkers using connectivity-based clustering from the diffusion-weighted imaging.

6. The method of claim 5, wherein the step of extracting diffusion-weighted imaging-derived values comprises:
calculating correlation coefficients between the diffusion-weighted imaging and biomarkers,
selecting a region with a correlation coefficient greater than a certain threshold, and
performing connectivity-based clustering by grouping connected voxels using relationships therebetween.

7. The method of claim 6, wherein the step of performing connectivity-based clustering comprises:
performing connectivity-based clustering by permuting the order between the diffusion-weighted imaging and biomarkers, and generating a null distribution using a number of voxels in the group with most abundant voxels as a statistic;
performing connectivity-based clustering without permuting the order between the diffusion-weighted imaging and the biomarkers, followed by extracting values of the voxels belonging to significant groups using the null distribution for each group; and
calculating a weighted sum of the extracted voxel values to create a score and training weights to maximize a correlation coefficient between the created score and the biomarkers.

8. The method of claim 3, wherein the step of predicting Alzheimer's disease-related biomarkers comprises:
calculating a biomarker-specific score using the diffusion-weighted imaging-derived values, the regional cortical thickness, and the trained weights, and
predicting the regional or global amyloid positivity or negativity using the calculated biomarker-specific score along with the demographic and clinical information.

9. The method of claim 1, wherein the step of training the biomarker prediction model comprises:
deriving a correlation between regional tau accumulation at a baseline time point and the cortical thickness of all individual regions at a follow-up time point using partial correlation analysis.

10. The method of claim 9, wherein the step of deriving a correlation using partial correlation analysis comprises:
deriving a correlation adjusted for the influence of demographic and clinical information used as covariates.

11. The method of claim 9, wherein the step of deriving a correlation using partial correlation analysis comprises:
deriving a correlation based on the progression of the disease, selecting regions with most consistent correlation with the tau accumulation of the target region, and
performing multi-output regression analysis based on the selected regions.

12. The method of claim 1, wherein the biomarker prediction model comprises:
a first prediction model that predicts tau accumulation at the baseline time point using the cortical thickness of selected regions at the follow-up time point and demographic and clinical information.

13. The method of claim 12, wherein the biomarker prediction model further comprises:
a second prediction model that predicts tau accumulation at the follow-up time point using the cortical thickness at the follow-up time point, the demographic and clinical information, and the tau accumulation at the baseline time point.

14. The method of claim 1, wherein the step of predicting Alzheimer's disease-related biomarkers comprises:
predicting tau accumulation at a past time point using the current cortical thickness and the demographic and clinical information.

15. The method of claim 14, wherein the step of predicting Alzheimer's disease-related biomarkers further comprises:
predicting tau accumulation at the current time point using the current cortical thickness, the demographic and clinical information, and the predicted tau accumulation at the past time point.

16. A computing device comprising:
a processor including one or more cores; and
memory,
wherein the processor is configured to collect medical images of a subject and preprocess the collected medical images to generate training data, train a biomarker prediction model using the generated training data and demographic and clinical information of the subject, wherein the training includes assigning weights to the training data, and predict Alzheimer's disease-related biomarkers for a patient using the trained biomarker prediction model.

17. A computer program stored on a computer-readable storage medium that includes instructions causing a computer to perform the following operations of:
collecting medical images of a subject and preprocessing the collected medical images to generate training data;
training a biomarker prediction model using the generated training data and demographic and clinical information of the subject, wherein the training includes assigning weights to the training data; and
predicting Alzheimer's disease-related biomarkers for a patient using the trained biomarker prediction model.
